(19) United States
(12) Patent Application Publication
(10) Pub. No.: US 2016/0331246 A1
(43) Pub. Date: Nov. 17, 2016

(54) Pulse diagnostic examination system and its instrument operation method

(71) Applicant: National Tsing Hua University, Hsinchu City (TW)

(72) Inventor: Swe-Kai CHEN, Hsinchu City (TW)

(21) Appl. No.: 14/818,880

(22) Filed: Aug. 5, 2015

(30) Foreign Application Priority Data
May 15, 2015 (TW) 104115469

(51) Int. Cl. A61B 5/024; A61B 5/02; A61B 5/026

(52) U.S. Cl. CPC A61B 5/024; A61B 5/026; A61B 5/02007

(57) ABSTRACT

The present disclosure relates to a pulse diagnostic examination system and an instrument operation method thereof. The system may include a pulse touching and sensing unit, a pulse frequency sensing unit, a pressure feedback sensing unit, a blood flow rate sensing unit, a carpi radial artery sensing and detecting unit, a data base unit and a data processing unit. The pulse touching and sensing unit is placed on nine touching and sensing points corresponding to cun, guan and chi of the arm of the human. The pulse touching and sensing unit may press and move along an artery vertically and horizontally corresponding to the nine points for detecting a pulse frequency, a downward-pressure feedback strength, a blood flow and a thickness of the artery. The result generated in the process may be compared with a pulse classification table for analyzing a corresponding pulse by the position, the strength, the frequency and the classification of the pulse for diagnose and treatment.

### BACKGROUND OF THE INVENTION

1. Field of the Invention

The present disclosure relates to a pulse diagnostic examination system and an instrument operation method thereof, and more specifically, to a pulse diagnostic examination system and instrument operation method thereof which have the function of preliminary pulse examination, can modulate the pulse examination of physicians, and digitalize the experiences of the pulse examination for education and the study of the same pulse examination.

2. Description of Related Art

In general, traditional Chinese physicians press a certain acupuncture point (i.e. cun, guan, chi and so forth) of a patient's wrist by their fingers. The traditional Chinese physicians could feel the pulse with three different strength of pressing for the superficial level, the mid-level and the deep level of the pulse. When the finger touches the skin softly without an exerting force, it is named as feeling the superficial level; when force is slightly exerted and the finger touches the position of muscle, it is named as feeling the mid-level; when force is further exerted and the finger touches the position of bone, it is named as feeling the deep level. Different strength of pressing can reflect the physical conditions of different organs, and different pulses can therefore be determined.

However, during the pulse examination, traditional Chinese physicians rely on their personal experience to exert different strengths for examining the pulse and determining the pulse. Different traditional Chinese physicians have different standards in the strength of pressing. Thus, they can only teach their students that different pulses would cause different feelings. Students are asked to practice even though they cannot determine the pulse based on their feelings. It might not become a serious problem in education. However, as a junior traditional Chinese physician, their varying determination of the pulse may not result in the most precise understanding with respect to the patient's condition, which may not have the corresponding properly treated likely worsening the condition of the patient.

In order to minimize the occurrence of the above condition, an alternative solution may be provided in below.

### SUMMARY OF THE INVENTION

According to the present disclosure, a pulse diagnostic examination system and an instrument operation method of the pulse diagnostic examination system are provided. The direction of the blood flow is defined as a first direction (horizontal direction), the radial direction of the artery is defined as a second direction (vertical direction), and a third direction is defined as being perpendicular to the second direction and being on the sectional surface of the artery. Thus, the pulses defined in traditional Chinese medicine can be located. Further, the pulse can be determined by considering the strength of the blood flow, the frequency of the pulse, and the shape.

According to one embodiment of the present disclosure, a pulse diagnostic examination system is provided. The pulse diagnostic examination system in one embodiment may include a pulse touching and sensing unit to be placed on an arm of a human corresponding to the direction of the blood flow. In one embodiment, the pulse touching and sensing unit may be placed on nine touching and sensing points of the arm of the human, wherein three of the nine touching and sensing points may correspond to the cun, other three of the nine touching and sensing points may correspond to the guan, and the remaining three of the nine touching and sensing points may correspond to chi. In one embodiment, the pulse touching and sensing unit may be connected with a vertical movement mechanism and a horizontal movement mechanism, wherein the vertical movement mechanism may be configured to cause the pulse touching and sensing unit to press the artery corresponding to the nine touching and sensing points vertically, and the horizontal movement mechanism may be configured to cause the pulse touching and sensing unit to move horizontally at the artery corresponding to the nine touching and sensing points. The disclosed system may include a pulse frequency sensing unit disposed on the pulse touching and sensing unit, and the pulse frequency sensing unit may be configured for detecting a pulse frequency of the nine touching and sensing points. The disclosed system may include a pressure feedback sensing unit disposed on the pulse touching and sensing unit may be configured for detecting a downward-pressure feedback strength of the nine touching and sensing points when the nine touching and sensing points may receive a downward-pressure from the vertical movement mechanism by different strength. Plus, the system may also include a blood flow rate sensing unit disposed on the pulse touching and sensing unit, with the blood flow rate sensing unit configured for detecting a blood flow of the artery when the horizontal movement mechanism may move horizontally at the artery corresponding to the nine touching and sensing points. A carpi radial artery sensing and detecting unit may be disposed on the pulse touching and sensing unit, the carpi radial artery sensing and detecting unit may be configured for detecting a thickness of the artery when the horizontal movement mechanism moves horizontally at the artery corresponding to the nine touching and sensing points. A data base unit having at least one pulse classification table may be included in the disclosed system. The pulse classification table may define a strength of blood flow, a frequency of the pulse and a classification of the pulse in advance. A data processing unit electrically connected with the vertical movement mechanism, the horizontal movement mechanism, the pulse frequency sensing unit, the pressure feedback sensing unit, the blood flow rate sensing unit and the data base unit may be also included in the system. The data processing unit may be configured for comparing the pulse frequency detected from the nine touching and sensing points by the pulse frequency sensing unit, the downward-pressure feedback strength detected from the nine touching and sensing points by the pressure feedback sensing unit and the blood flow detected from the nine touching and sensing points by the blood flow rate sensing unit with the pulse classification table in the data base unit for determining the corresponding pulse.

More specifically, the vertical movement mechanism is capable of determining a plurality of distances of the vertical and downward movement, the data processing unit is capable of analyzing the pulse according to the distances of the vertical and downward movement, the downward-pressure feedback strength detected by the pressure feedback sensing unit and the pulse frequency detected by the pulse frequency sensing unit.

More specifically, based on the downward-pressure feedback strength detected by the pressure feedback sensing unit, the pulse of each of the nine touching and sensing points may be determined as having a pulse, having no pulse, a powerful pulse, or a powerless pulse so as to analyze the pulse.

More specifically, based on the downward-pressure feedback strength detected by the pressure feedback sensing unit, which of the nine touching and sensing points having a pulse may be determined, and the pulse may be determined as a long pulse or a short pulse.

More specifically, based on the pulse frequency detected by the pulse frequency sensing unit, the pulse may be determined as regular or irregular so as to analyze the pulse.

More specifically, the horizontal movement mechanism is capable of moving horizontally corresponding to the nine touching and sensing points for detecting the pulse based on the blood flow detected by the blood flow rate sensing unit and the thickness of the artery detected by the carpi radial artery sensing and detecting unit so as to analyze the pulse.

More specifically, based on the blood flow detected by the blood flow rate sensing unit, the blood flow may be determined as fluent or influent so as to analyze the pulse.

More specifically, based on the thickness of the artery detected by the carpi radial artery sensing and detecting unit when moving along the artery horizontally, the pulse may be determined as a thick pulse or a thin pulse so as to analyze the pulse.

According to another embodiment of the disclosure, an instrument operation method of a pulse diagnostic examination system is provided. The instrument operation method of a pulse diagnostic examination system may include the following steps: (1) placing a pulse touching and sensing unit on an arm of a human corresponding to the direction of the blood flow, and placing the pulse touching and sensing unit on nine touching and sensing points of the arm of the human, wherein three of the nine touching and sensing points correspond to cun, other three of the nine touching and sensing points correspond to guan, and the remaining three of the nine touching and sensing points correspond to chi, (2) moving the pulse touching and sensing unit by a vertical movement mechanism and a horizontal movement mechanism, such that the pulse touching and sensing unit pressing an artery corresponding to the nine touching and sensing points vertically with different pressures and the pulse touching and sensing unit moves horizontally at the artery corresponding to the nine touching and sensing points, (3) obtaining a pulse frequency, a downward-pressure feedback strength, a blood flow of an artery and a thickness of the artery by a plurality of sensing units disposed on the pulse touching and sensing unit, and (4) comparing the pulse frequency, the downward-pressure feedback strength, the blood flow of an artery and the thickness of the artery with their counterparts in a pulse classification table for analyzing a corresponding pulse.

More specifically, based on the downward-pressure feedback strength, the pulse of each of the nine touching and sensing points may be be determined as having a pulse, having no pulse, a powerful pulse, or a powerless pulse so as to analyze the pulse.

More specifically, based on the downward-pressure feedback strength, which of the nine touching and sensing points having a pulse may be determined, and the pulse may be determined as a long pulse or a short pulse.

More specifically, based on the pulse frequency, the pulse may be determined as regular or irregular so as to analyze the pulse.

More specifically, based on the blood flow, the blood flow may be determined as fluent or influent so as to analyze the pulse.

More specifically, based on the thickness of the artery detected when certain component of the disclosed system moves along the artery horizontally, the pulse may be determined as a thick pulse or a thin pulse so as to analyze the pulse.

### BRIEF DESCRIPTION OF THE DRAWINGS

FIG. 1 is a schematic view of a pulse diagnostic examination system of the disclosure according to one embodiment of the present disclosure.

FIG. 2 is a flow chart of an instrument operation method of a pulse diagnostic examination system according to one embodiment of the present disclosure.

FIG. 3 is a basic pulse classification table of the disclosure.

FIG. 4 is a complex pulse classification table of the disclosure.

### DETAILED DESCRIPTION OF THE PREFERRED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

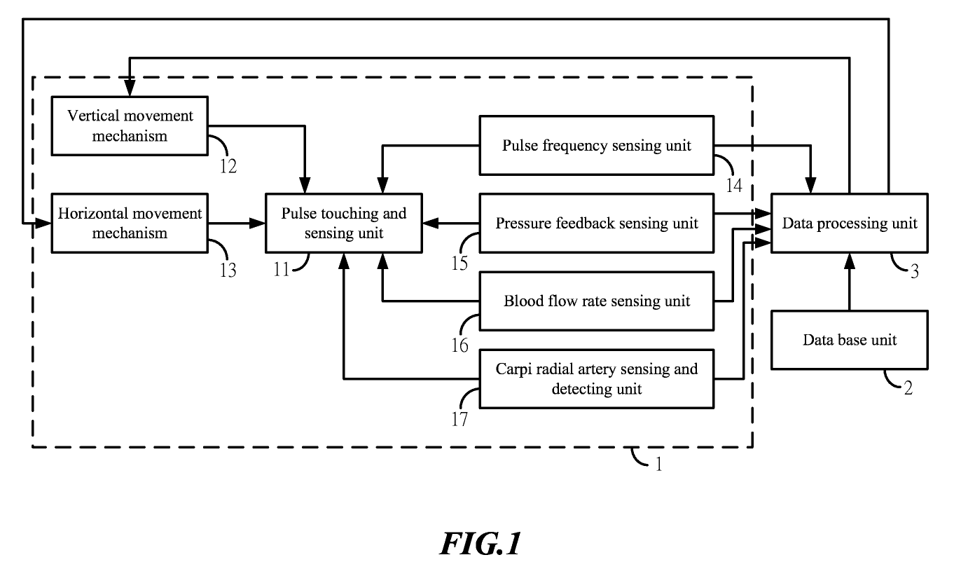

Referring to FIG. 1, FIG. 1 is a schematic view of a pulse diagnostic examination system according to one embodiment of the present disclosure. As shown in FIG. 1, the pulse diagnostic examination system may include a pulse touching and sensing unit 1, a data processing unit 3, and a data base unit 2. The pulse touching and sensing unit 1 may further include a touching and sensing assembly 11, which may be configured for touching an arm of a human body. The sensing assembly 11 may be designed based on seven examinations (top, middle, bottom, high, low, left and right) proposed by a famous traditional Chinese physicians, Li, Zhong Zi (Ming dynasty to Qing dynasty) as well as principles of four examinations in Yil Zongl Jinl Jian. Li, Zhong Zi proposed that “each of cun, guan and chi can be further divided into three parts, so that cun, guan and chi have totally nine parts. Thus, the position of “first part of guan” should be considered as within guan, i.e. the front part of the three parts of guan, not cun, which is in front of guan. Accordingly, Li held the view that ren-ying and qi kou are located at the left (i.e. left hand) guan and the right (i.e. right hand) guan, respectively. The position of first part of the left guan indicates liver, which is the viscus of wind and wood. Thus, when one is hurt by wind, their viscus of wind and liver pulse would be correspondingly prosperous. The position of first part of the right guan indicates spleen, which is earth of middle and office of the granaries. Thus, when one is hurt by food, their viscus of earth and spleen pulse would be correspondingly prosperous.

Therefore, when in use the nine pressing mechanisms (in another embodiment the number of the pressing mechanism may be fewer than nine) of the pulse touching and sensing unit 11 may align with the direction of the blood flow and the nine touching and sensing points corresponding to cun, guan and chi (cun, guan, chi, the positions in front of them and the positions behind them, i.e. nine touching and sensing points; three touching and sensing points corresponding to cun, guan and chi are firstly set, then the positions in front of them and the positions behind them are set, such that there are totally nine touching and sensing points). The alignment may be accomplished by hand or by an additional alignment device disposed in the pulse diagnostic examination system.

The pulse touching and sensing unit 1 may be connected with a vertical movement mechanism 12 and a horizontal movement mechanism 13. The vertical movement mechanism 12 may be configured for forcing the pulse touching and sensing unit 11 to press an artery corresponding to the nine touching and sensing points vertically. The horizontal movement mechanism 13 may be configured for forcing the pulse touching and sensing unit 11 to move horizontally at the artery corresponding to the nine touching and sensing points.

Since the famous traditional Chinese physicians, Li, Zhong Zi (Ming dynasty to Qing dynasty) proposed the concept of 50-times-examination, which means one should take the pulse 50 times for each of the touching and sensing point. Thus, the data processing unit 3 may be used for controlling the vertical movement mechanism 12 and the horizontal movement mechanism 13 so as to press (and move horizontally) the touching and sensing point 50 times, which cannot be accomplished in the conventional approach.

Moreover, the pulse touching and sensing unit 1 may further include a pressure feedback sensing unit 15, a pulse frequency sensing unit 14, a blood flow rate sensing unit 16 and a carpi radial artery sensing and detecting unit 17. The pressure feedback sensing unit 15 may be configured for detecting a downward-pressure feedback strength of the nine touching and sensing points when the nine touching and sensing points receives a downward-pressure from the vertical movement mechanism by different strength so as to analyze the pulse. Furthermore, based on the downward-pressure feedback strength, which of the nine touching and sensing points having a pulse may be determined, and the pulse can be determined as a long pulse or a short pulse.

In addition, based on the pulse frequency detected by the pulse frequency sensing unit 14, the pulse may be determined as regular or irregular so as to analyze the pulse.

In addition, based on the blood flow detected by the blood flow rate sensing unit 16, the blood flow may be determined as fluent or influent so as to analyze the pulse.

In addition, based on the thickness of the artery detected by the carpi radial artery sensing and detecting unit 17 moving along the artery horizontally, the pulse may be determined as a thick pulse or a thin pulse so as to analyze the pulse.

In addition, the data base unit 2 may be equipped with at least one pulse classification table. A strength of blood flow, a frequency of the pulse and a classification of the pulse may be defined in the pulse classification table in advance. Thus, the data processing unit 3 may compare the pulse frequency, the downward-pressure feedback strength and the blood flow detected from the nine touching and sensing points with the pulse classification table in the data base unit 2 for determining the corresponding pulse.

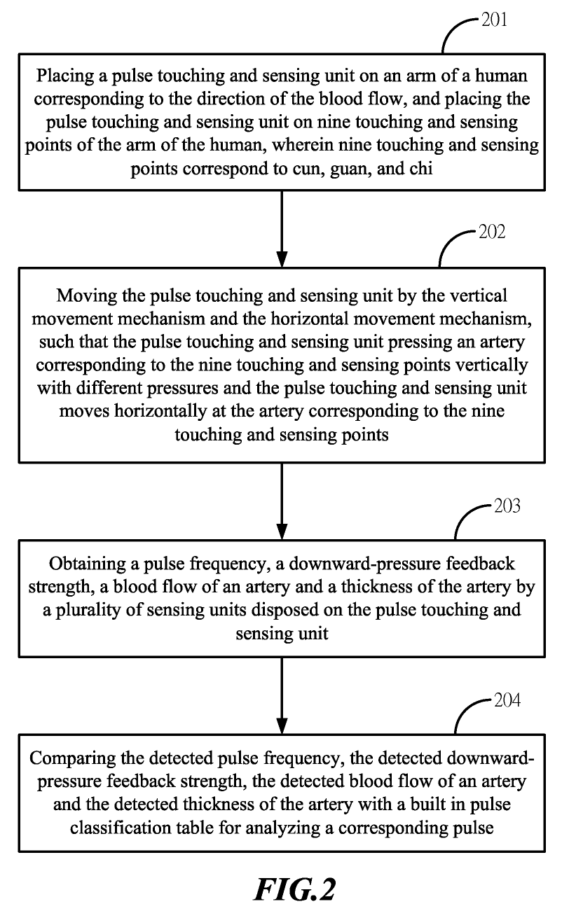

According to another embodiment of the disclosure, an instrument operation method of a pulse diagnostic examination system is provided. As shown in FIG. 2, the instrument operation method of a pulse diagnostic examination system may include the following steps:

Step 201: placing a pulse touching and sensing unit on an arm of a human corresponding to the direction of the blood flow, and placing the pulse touching and sensing unit on nine touching and sensing points of the arm of the human, wherein nine touching and sensing points correspond to cun, guan, and chi;

Step 202: moving the pulse touching and sensing unit by the vertical movement mechanism and the horizontal movement mechanism, such that the pulse touching and sensing unit pressing an artery corresponding to the nine touching and sensing points vertically with different pressures and the pulse touching and sensing unit moves horizontally at the artery corresponding to the nine touching and sensing points;

Step 203: obtaining a pulse frequency, a downward-pressure feedback strength, a blood flow of an artery and a thickness of the artery by a plurality of sensing units disposed on the pulse touching and sensing unit;

Step 204: comparing the detected pulse frequency, the detected downward-pressure feedback strength, the detected blood flow of an artery and the detected thickness of the artery with their counterparts in the pulse classification table for analyzing a corresponding pulse.

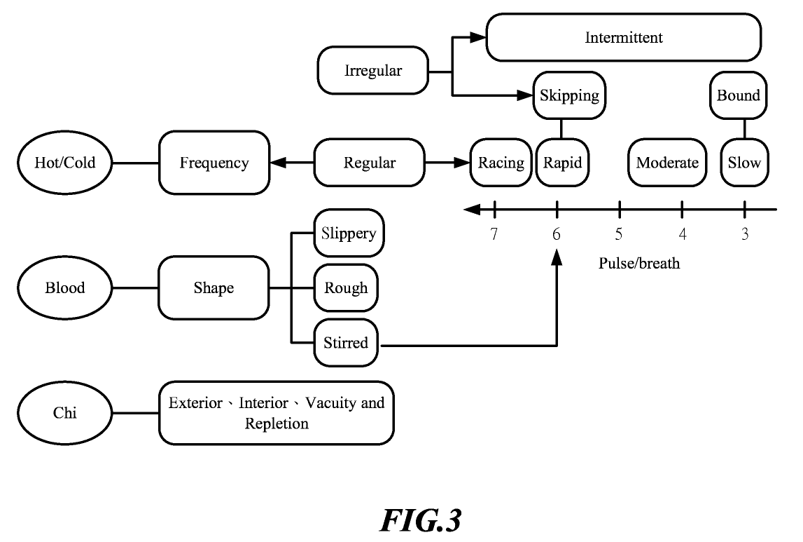

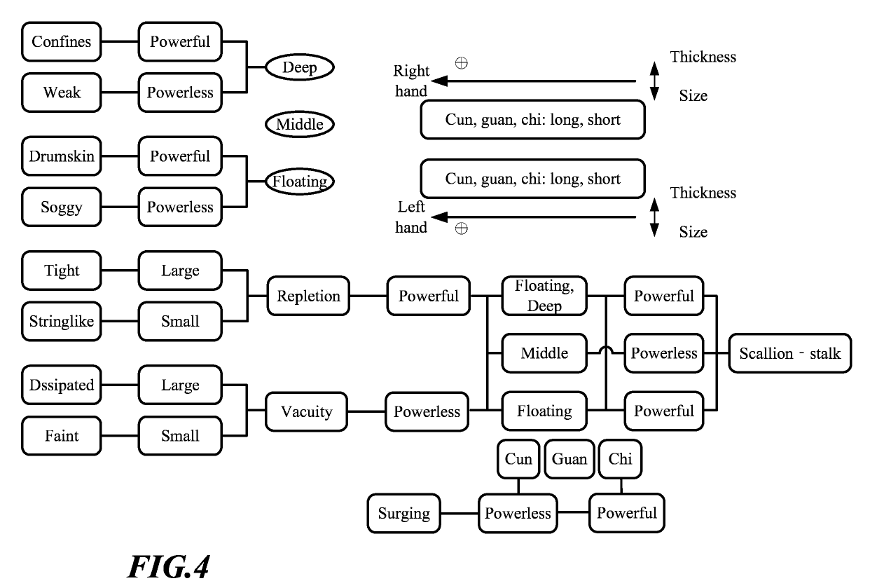

As shown in FIGS. 3 and 4, a basic pulse classification table and a complex pulse classification table of one embodiment of the present disclosure are provided. Pulses may be classified according to “frequency” and “shape.” “Exterior, interior, vacuity and repletion” are used to study the pathology. In general, basic pulses can be classified as floating pulse, middle pulse, deep pulse, hidden pulse, large pulse, small pulse, fine pulse, long pulse, short pulse, slippery pulse, rough pulse, vacuous pulse, replete pulse, slow pulse, rapid pulse, moderate pulse, racing pulse, bound pulse, skipping pulse and intermittent pulse. “Floating pulse, middle pulse, deep pulse and hidden pulse” are determined by the applicable distance of the vertical and downward motion (four ranges of the motion are set up, such that the relative depth of floating pulse, middle pulse, deep pulse and hidden pulse can be defined). Further, the pressure feedback sensing unit 15 may detect the shortest vertical distance by which the pulse may be detected. According to the definition of the floating pulse and the deep pulse, “the one which can be detected near the skin is the floating pulse, and the one which can be detected near the supplement sinew and bone is the deep pulse.” Accordingly, if the pulse is detected within a short distance, the pulse may be determined as a floating pulse. On the other hand, if the pulse can be detected only within a long distance, the pulse may be determined as a deep pulse. Thus, the distance can be set up so as to define the floating pulse, the middle pulse, the deep pulse and the hidden pulse. Therefore, a pulse classification table can be built up in the data base unit 2, such that the relationship between the distance and the floating pulse, the middle pulse, the deep pulse and the hidden pulse is defined. Thereby, when the distance falls into a certain range, then the pulse may be automatically determined as a floating pulse, a middle pulse, a deep pulse or a hidden pulse.

According to the definition of the pulse examination, “the one having a large size is a large pulse while the one having a small size is a small pulse, i.e. a fine pulse.” According to the present disclosure, the carpi radial artery sensing and detecting unit 17 may detect a thickness of the artery when the pulse touching and sensing unit 11 moves horizontally at the artery corresponding to the nine touching and sensing points, such that the pulse may be determined as a thick pulse or a thin pulse, and the pulse may further be determined as a large pulse or a small pulse. Thus, a pulse classification table is further built up in the data base unit 2, such that the thickness of the large pulse and the small pulse may be defined. Thereby, when the thickness of the pulse falls into a certain range, then the pulse may be automatically determined as a large pulse or a small pulse.

According to the definition of the pulse examination, “the one traveling with a long distance is a long pulse and the one traveling with a short distance is a short pulse.” According to the present disclosure, the pressure feedback sensing unit 15 may detect a downward-pressure feedback strength. Thus, when the touching and sensing point is pressed, which of the nine touching and sensing points having a pulse can be determined based on the downward-pressure feedback strength. If a few of the touching and sensing points have a pulse, or the lines extending from the nine touching and sensing points also have a pulse, it may be categorized as a long pulse. Otherwise, it may be determined as a short pulse. Thus, a pulse classification table may be further built up in the data base unit 2, such that the length of the long pulse and the short pulse may be defined. Thereby, when the length of the pulse falls into a certain range, then the pulse can be automatically determined as a long pulse or a short pulse.

According to the definition of the pulse examination, “the one which can move slipperily is a slippery pulse and the one which hardly moves is a rough pulse.” According to the present disclosure, the blood flow rate sensing unit 16 may detect a blood flow of the artery. Thus, based on the blood flow, the blood flow may be determined as fluent or influent. If the detected blood flow is slow, it may be considered as a rough pulse; otherwise, one preliminary conclusion of a slippery pulse may be reached. Thus, a pulse classification table may be further built up in the data base unit 2, such that the relationship between the rate of the blood flow and the rough pulse and the slippery pulse may be defined. Thereby, when the rate of the blood flow falls into a certain range, then the pulse may be automatically determined as a rough pulse or a slippery pulse.

According to the definition of the pulse examination, “when all of floating pulse, middle pulse and deep pulse are powerful, it is considered as replete pulse and when all of the floating pulse, middle pulse and deep pulse are powerless, it is determined as vacuous pulse.” When it is pressed by differences corresponding to the floating pulse, middle pulse and deep pulse, based on the downward-pressure feedback strength detected by the pressure feedback sensing unit 15, the pulse may be considered as having a pulse, having no pulse, a powerful pulse, or a powerless pulse. Thus, a pulse classification table may be further built up in the data base unit 2, such that the relationship between the downward-pressure feedback strength and having a pulse, having no pulse, the powerful pulse and the powerless pulse is defined. Thereby, the pulse may be automatically categorized as having a pulse, having no pulse, the powerful pulse and the powerless pulse. Further, the floating pulse, middle pulse and deep pulse may be regarded as a powerful pulse or a powerless pulse, such that it may be considered as a vacuous pulse or a replete pulse.

According to the definition of the pulse examination, “one breath includes breath-in and breath-out and when there are three pulses within a breath, it may be branded as the slow pulse and when there are six pulses within a breath, it may be considered as the rapid pulse.” The nomenclature may be based on the number of pulses within a breath. “When there are four pulses within a breath, it may be considered as a moderate pulse. When there are seven pulses within a breath, the conclusion of a racing pulse may be reached.” According to the present disclosure, the blood flow rate sensing unit 16 may detect a blood flow of the artery. According to the present disclosure, the pulse frequency sensing unit 14 may detect a pulse frequency. Based on the pulse frequency, it may be determined as a slow pulse, a rapid pulse, a moderate pulse or a racing pulse. Thus, a pulse classification table may be further built up in the data base unit 2, such that the relationship between the pulse frequency and the slow pulse, the rapid pulse, the moderate pulse and the racing pulse may be defined. Thereby, the pulse may be automatically regarded as a slow pulse, a rapid pulse, a moderate pulse or a racing pulse.

According to the definition of the pulse examination, “if there is one stop in a moderate pulse, a bound pulse may be present and if there is one stop in a rapid pulse, a skipping pulse may be present.” Plus, the bound pulse and the skipping pulse can recover after stopping. “If it stops and recovers after a while, such that there is one stop in ten, twenty or even thirty pulses, an intermittent pulse may be present.” The definitions of the slow pulse, the rapid pulse, the moderate pulse and the racing pulse may be categorized based on the times of pulses within a certain period of time, and the times of pulses within a certain period of time is regular. However, the bound pulse and the skipping pulse may be associated with a sudden stop between the pulses. Thus, a pulse classification table is further built up in the data base unit 2, such that it can be considered as a bound/skipping pulse when there is a stop in four/six pulses, and it can be considered as intermittent pulse when the stop is irregular.

A complex pulse includes “a surging pulse, a stringlike pulse, a tight pulse, a soggy pulse, a weak pulse, a drumskin pulse, a confines pulse, a faint pulse and a dissipated pulse.” According to the definition of the pulse examination, “a surging pulse is that it becomes stronger when pressed by a finger and becomes weaker when the finger departs.” Thus, a pulse classification table may be further built up in the data base unit 2, such that when the downward-pressure feedback strength detected from the touching and sensing points corresponding to can may be considered as a powerless pulse, and the downward-pressure feedback strength detected from the touching and sensing points corresponding to chi is determined as a powerful pulse, it may be automatically categorized as a surging pulse.

According to the definition of the pulse examination, “a string-like pulse is that its shape is like a string, it is thin and straight, with a strong force.” A tight pulse is that it is thicker than the string-like pulse with a strong and bouncing force. Thus, a pulse classification table may be further built up in the data base unit 2, such that when a replete pulse is present, it may be further determined as a thick pulse or a thin pulse. If the artery is determined as a thick pulse, it may be considered also as a tight pulse; in contrast, if the artery is determined as a thin pulse, it may be further regarded as a string-like pulse.

According to the definition of the pulse examination, “a soggy pulse may be floating and powerless, a weak pulse may be deep and powerless, a drumskin pulse may be floating and very powerful, and a confines pulse may be deep and very powerful.” Thus, a pulse classification table may be further built up in the data base unit 2, such that when a floating pulse is present, it may be further determined as a powerful pulse or a powerless pulse based on the downward-pressure feedback strength. If a powerful pulse is present, it may be further determined as a drumskin pulse; in contrast, if a drumskin pulse is present, it may be further categorized as a soggy pulse; in addition, a pulse classification table may be further built up in the data base unit 2, such that when a deep pulse is present, it may be further determined as a powerful pulse or a powerless pulse based on the downward-pressure feedback strength. If it is determined as a powerful pulse, it may be as a confines pulse; in contrast, if it is determined as a weak pulse, it may be a soggy pulse.

According to the definition of the pulse examination, “a faint pulse is that when the floating pulse, the middle pulse and the deep pulse are very powerless, and the pulse is very small and may not be really felt when measured.” “A dissipated pulse is that when the floating pulse, the middle pulse and the deep pulse are very powerless though the pulse is very large with an enlarged artery.” Thus, a pulse classification table may be further built up in the data base unit 2, such that when it is determined as a vacuous pulse, it may be further determined as a thick pulse or a thin pulse. If the pulse is determined as a thick pulse, it may be considered as a dissipated pulse; in contrast, if the pulse is determined as a thin pulse, it may be further determined as a faint pulse.

Besides the above described basic pulses and complex pulses, there are other pulses, such as the scallion-stalk pulse and the stirred pulse. According to the definition of the pulse examination, “a scallion-stalk pulse is that the floating pulse and the deep pulse are powerful while the middle pulse is powerless.” Thus, a pulse classification table is further built up in the data base unit 2, such that when the floating pulse and the deep pulse are determined as powerful pulses while the middle pulse is determined as a powerless pulse, it may be regarded as a scallion-stalk pulse. According to the definition of the pulse examination, “a stirred pulse is that the shape is like a bean and it beats irregularly.” Thus, when the pulse is determined as lack of regular beats (without stopping), it may be categorized as a stirred pulse.

In some embodiments, the data processing unit 3 may be further connected with a display device (not shown in the figure). Thus, after the classification of the pulse is determined by the data processing unit 3 according to the pulse classification table in the data base unit 2, the result can be directly shown in the display screen. Thereby, the user can reference the result for their diagnosis and treatment.

According to the pulse diagnostic examination system and the instrument operation method of the present disclosure, as compared with conventional technique, the disclosure has the following advantages:

1. According to the present disclosure, the direction of the blood flow is defined as a first direction (horizontal direction), the radial direction of the artery is defined as a second direction (vertical direction), and a third direction is defined as being perpendicular to the second direction and being on the sectional surface of the artery. Thus, the pulses defined in traditional Chinese medicine can be located. Further, the pulse can be determined by considering the strength of the blood flow, the frequency of the pulse, and the shape.

2. The present disclosure provides a pulse classification table. The pulse classification table may set up a plurality of conditions of pulses. When the detection is congruent with the conditions, the pulse can be automatically determined and categorized.

According to the above descriptions, the actuator used for an electric motor of the disclosure can achieve the purposes and solve the problems met in conventional technologies. Thus, the disclosure involves novelty and inventiveness as well as meets the requirements of patent application. Thus, the application is applied according to Patent Law. The application is kindly requested to be granted.

What is claimed is:

1. A pulse diagnostic examination system, comprising:
a pulse touching and sensing unit, placed on an arm of a human corresponding to a direction of a blood flow, wherein the pulse touching and sensing unit is placed on nine touching and sensing points of the arm of the human, with three of the nine touching and sensing points corresponding to cun, other three of the nine touching and sensing points corresponding to guan, and the remaining three of the nine touching and sensing points corresponding to chi, the pulse touching and sensing unit is connected with a vertical movement mechanism and a horizontal movement mechanism, the vertical movement mechanism is configured for causing the pulse touching and sensing unit to press an artery corresponding to the nine touching and sensing points vertically, and the horizontal movement mechanism is configured for causing the pulse touching and sensing unit to move horizontally at the artery corresponding to the nine touching and sensing points;
a pulse frequency sensing unit, disposed on the pulse touching and sensing unit, wherein the pulse frequency sensing unit is configured for detecting a pulse frequency of the nine touching and sensing points;
a pressure feedback sensing unit, disposed on the pulse touching and sensing unit, wherein the pressure feedback sensing unit is configured for detecting a downward-pressure feedback strength of the nine touching and sensing points when the nine touching and sensing points receives a downward-pressure from the vertical movement mechanism by different strength;
a blood flow rate sensing unit, disposed on the pulse touching and sensing unit, wherein the blood flow rate sensing unit is configured for detecting the blood flow of the artery when the horizontal movement mechanism moves horizontally at the artery corresponding to the nine touching and sensing points;
a carpi radial artery sensing and detecting unit, disposed on the pulse touching and sensing unit, wherein the carpi radial artery sensing and detecting unit is configured for detecting a thickness of the artery when the horizontal movement mechanism moves horizontally at the artery corresponding to the nine touching and sensing points;
a data base unit, having at least one pulse classification table in which a strength of the blood flow, a frequency of the pulse and a classification of the pulse are defined in advance; and
a data processing unit, electrically connected with the vertical movement mechanism, the horizontal movement mechanism, the pulse frequency sensing unit, the pressure feedback sensing unit, the blood flow rate sensing unit and the data base unit, wherein the data processing unit is configured for comparing the pulse frequency detected from the nine touching and sensing points by the pulse frequency sensing unit, the downward-pressure feedback strength detected from the nine touching and sensing points by the pressure feedback sensing unit and the blood flow detected from the nine touching and sensing points by the blood flow rate sensing unit with the pulse classification table in the data base unit for determining the corresponding pulse.

2. The pulse diagnostic examination system according to claim 1, wherein the vertical movement mechanism is capable of determining a plurality of distances of the vertical and downward movement, and the data processing unit is capable of analyzing the pulse according to the distances of the vertical and downward movement, the downward-pressure feedback strength detected by the pressure feedback sensing unit and the pulse frequency detected by the pulse frequency sensing unit.

3. The pulse diagnostic examination system according to claim 2, wherein based on the downward-pressure feedback strength detected by the pressure feedback sensing unit, the pulse of each of the nine touching and sensing points is determined as having a pulse, having no pulse, a powerful pulse, or a powerless pulse so as to analyze the pulse.

4. The pulse diagnostic examination system according to claim 2, wherein based on the downward-pressure feedback strength detected by the pressure feedback sensing unit, which of the nine touching and sensing points having a pulse is determined, and the pulse is determined as a long pulse or a short pulse.

5. The pulse diagnostic examination system according to claim 2, wherein based on the pulse frequency detected by the pulse frequency sensing unit, the pulse is determined as regular or irregular so as to analyze the pulse.

6. The pulse diagnostic examination system according to claim 1, wherein the horizontal movement mechanism is capable of moving horizontally corresponding to the nine touching and sensing points for detecting the pulse based on the blood flow detected by the blood flow rate sensing unit and the thickness of the artery detected by the carpi radial artery sensing and detecting unit so as to analyze the pulse.

7. The pulse diagnostic examination system according to claim 6, wherein based on the blood flow detected by the blood flow rate sensing unit, the blood flow is determined as fluent or influent so as to analyze the pulse.

8. The pulse diagnostic examination system according to claim 6, wherein based on the thickness of the artery detected by the carpi radial artery sensing and detecting unit moving along the artery horizontally, the pulse is determined as a thick pulse or a thin pulse so as to analyze the pulse.

9. An instrument operation method of a pulse diagnostic examination system, comprising:
placing a pulse touching and sensing unit on an arm of a human corresponding to a direction of a blood flow, and placing the pulse touching and sensing unit on nine touching and sensing points of the arm of the human, wherein three of the nine touching and sensing points correspond to cun, other three of the nine touching and sensing points correspond to guan, and the remaining three of the nine touching and sensing points correspond to chi;
moving the pulse touching and sensing unit by a vertical movement mechanism and a horizontal movement mechanism, such that the pulse touching and sensing unit presses an artery corresponding to the nine touching and sensing points vertically with different pressures and the pulse touching and sensing unit moves horizontally at the artery corresponding to the nine touching and sensing points;
obtaining a pulse frequency, a downward-pressure feedback strength, the blood flow of an artery and a thickness of the artery by a plurality of sensing units disposed on the pulse touching and sensing unit;
comparing the pulse frequency, the downward-pressure feedback strength, the blood flow of an artery and the thickness of the artery with a built in pulse classification table for analyzing a corresponding pulse.

10. The instrument operation method of a pulse diagnostic examination system according to claim 9, wherein based on the downward-pressure feedback strength, the pulse of each of the nine touching and sensing points is determined as having a pulse, having no pulse, a powerful pulse, or a powerless pulse so as to analyze the pulse.

11. The instrument operation method of a pulse diagnostic examination system according to claim 9, wherein based on the downward-pressure feedback strength, which of the nine touching and sensing points having a pulse is determined, and the pulse is determined as a long pulse or a short pulse.

12. The instrument operation method of a pulse diagnostic examination system according to claim 9, wherein based on the pulse frequency, the pulse is determined as regular or irregular so as to analyze the pulse.

13. The instrument operation method of a pulse diagnostic examination system according to claim 9, wherein the blood flow is determined as fluent or influent so as to analyze the pulse.

14. The instrument operation method of a pulse diagnostic examination system according to claim 9, wherein based on the thickness of the artery detected when the pulse diagnostic examination system moves along the artery, the pulse is determined as a thick pulse or a thin pulse so as to analyze the pulse.
